(19) United States
(12) Patent Application Publication
(10) Pub. No.: US 2026/0248382 A1
(43) Pub. Date: Aug. 27, 2026

(54) QUALITY ASSURANCE IN MEDICAL FACIAL IMAGES WITH RESPECT TO AMBIENT ILLUMINATION

(71) Applicant: KONTIGO CARE AB, Uppsala (SE)

(72) Inventors: Markku HÄMÄLÄINEN, UPPSALA (SE); Andreas ZETTERSTRÖM, MÅNKARBO (SE); Karl ANDERSSON, Vänge (SE)

(21) Appl. No.: 18/875,884

(22) PCT Filed: Jun. 21, 2023
(86) PCT No.: PCT/SE2023/050638

§ 371 Date: Dec. 17, 2024

(30) Foreign Application Priority Data
Jun. 23, 2022 (SE) 2250778-4

(51) Int. Cl. A61B 3/11; A61B 3/00; A61B 3/113; A61B 3/14

(52) U.S. Cl. CPC A61B 3/11; A61B 3/0025; A61B 3/113; A61B 3/14

(57) ABSTRACT

A user-operated hand-held medical body-imaging device (10) comprises a primary camera (20), user-interaction means (30), data communication means (40) and processing means (50). The primary camera is configured for capturing imaging data of a face of a user and for providing pre-capturing image information, comprising ambient illumination level, as experienced in a first direction. The user-interaction means is configured for providing information to the user. The data communication means is configured for external data communication. The processing means is configured for receiving the pre-capturing image information from the primary camera, for evaluating if acceptable conditions for capturing medically analyzable image data are present based on the pre-capturing image information, and for communicating operation instructions to the user in response to the evaluation. The processing means is further configured for recording the imaging data from the primary camera and for transmitting data representing the imaging data using the data communication means.

### TECHNICAL FIELD

The present invention relates in general to devices and methods for capturing images of body parts and in particular to handheld, self-operated devices and methods for capturing images of faces.

### BACKGROUND

One type of body imaging is pupillometry. Pupillometry, the study of pupils and their reactions to stimuli, has been used by medical practitioners for many decades. One relevant historic disclosure illustrating this is the published U.S. Pat. No. 3,966,310.

Devices for monitoring pupil size and pupil responsiveness characteristics are well known in the art. They are generally referred to as pupillometry systems or, simply, pupilometers. Handheld systems for measuring the pupillary response to light stimulus pulses have also been described in the past. The published U.S. Pat. No. 6,116,736 discloses a handheld pupillometry device which includes an imaging sensor, light emitters capable of illuminating the eye at different intensities with infrared (IR), yellow and blue coloured light, a battery powered image signal processing board and a display for which a graphical user interface is implemented to communicate with the user.

The device described in U.S. Pat. No. 6,116,736 can detect the pupillary response to light stimulus in a time resolved manner, for example by activating and deactivating visible for successive 1 second intervals for a period of 10 seconds total while transferring image frames at a rate set to 50 frames per second. The captured data can undergo feature extraction by use of several image processing procedures that are used to isolate a pupil within an image and to extract several pupil features such as size, shape and position from each pupil image data frame. An example of an extracted feature is the fitted radius and centre of pupil, calculated for 48 radii. Most processing procedures are performed on each image data frame, with the exception of an automatic thresholding procedure. The automatic thresholding procedure is applied during an initial calibration phase and, therefore, does not need to be applied to each image data frame. The thresholding function automatically identifies a grey level value that separates the pupil from the background in an image data frame. Moreover, when an appropriate threshold value is determined, all pixels having a grey level value greater than the threshold value are considered to comprise part of the image of the pupil, and all pixels having a grey level value less than the threshold are considered to correspond to background. A series of image frames captured during light exposure and for which features have been extracted can further be analysed to provide estimates of latency of pupil response to light stimulus, and pupil constriction velocity, to mention two examples. The results of the analyses can be used to arrive at one or more scalar values that are indicative of an overall physiologic or pathologic condition of the patient or, alternatively, to arrive at one or more scalar values that are indicative of an overall opto-neurologic condition of the patient.

With the advent of the smartphone-a handheld device designed that includes a telephone, computer, camera and many other sensors and actuators-attempts to use a smartphone as a pupillometer has been made. Kim and Youn describes one such attempt in the published article “Development of a Smartphone-based Pupillometer”, Journal of the Optical Society of Korea, Vol. 17, No. 3, June 2013, pp. 249-254. Later, refined smartphone-based systems have been described, for example in the published international patent application WO 2021/037788 A1. Use of smartphone-based pupilometers has been advised against by Mckay and co-authors in the publication “Evaluation of two portable pupillometers to assess clinical utility”, Concussion (2020) 5(4), CNC82, https://doi.org/10.2217/cnc-2020-0016, because the tested smartphone application did not produce concordant results to a validated desktop pupilometer.

A review article “Pupillary light reflex as a diagnostic aid from computational viewpoint: A systematic literature review”, by Hedenir Monteiro Pinheire and Ronaldo Martins da Costa, J. Biomedical Informatics 117 (2021) 103757, lists a number of pupillometry devices. The types of devices that were considered as reliable did not comprise any smart-phone based equipment. The reason is probably that such implementations still present too large variations in the imaging quality. As mentioned in “The Effect of Ambient Light Conditions on Quantitative Pupillometry”, by C. Ong et al, Neurocrit Care (2019) 30:316-321, in order to produce valid results and with maximum reliability, examiners should standardize the brightness of the environment, which is difficult to obtain with handheld self-operated devices, e.g. implemented in smart phones.

Within the field of telemedicine, a monitoring device with structured illumination has been disclosed in US 2015/0119652 A1. This monitoring device comprises an imaging system and an illumination system. The illumination is adapted to make the monitoring device produce images of a quality suitable for medical purposes. The evaluation of the illumination and the issue of a light control signal to the illumination system is performed by a second unit, with which the monitoring device communicates. Such a solution is therefore dependent on the provision of a remotely controllable illumination system.

An improvement of devices and methods for handheld self-operated devices for pupillometry or other body imaging processes used in ambient light conditions is therefore requested for giving more reliable image properties.

### SUMMARY

A general object of the present technology is to provide methods and devices giving more reliable image properties in body image capturing.

The above object is achieved by methods and devices according to the independent claims. Preferred embodiments are defined in dependent claims.

In general words, in a first aspect, a user-operated hand-held medical body-imaging device comprises a primary camera, user-interaction means, data communication means and processing means. The primary camera is configured for capturing imaging data of at least a part of a face of a user and for providing pre-capturing image information as experienced in a first direction. The imaging data comprises at least one of an image and a video stream. The pre-capturing image information comprises ambient illumination level as experienced in the first direction. The user-interaction means is configured for providing information to the user. The data communication means is configured for external data communication. The processing means is connected to the primary camera, the user-communication means and the data communication means. The processing means is configured for receiving the pre-capturing image information from the primary camera, for evaluating if acceptable conditions for capturing medically analyzable image data are present based on the pre-capturing image information, and for communicating operation instructions to the user in response to the evaluation, using the user-communication means. The processing means is further configured for recording the imaging data from the primary camera and for transmitting data representing the imaging data using the data communication means.

In a second aspect, a method for analyzing a face of a user comprises providing, in a user-operated hand-held medical body-imaging device, of pre-capturing image information. The pre-capturing image information comprises ambient illumination level as experienced in the first direction towards the user. In the user-operated hand-held medical body-imaging device, it is evaluated whether acceptable conditions for capturing medically analyzable image data are present, based on the pre-capturing image information. Operation instructions are communicated from the user-operated hand-held medical body-imaging device to the user in response to the evaluation. In the user-operated hand-held medical body-imaging device, imaging data of at least a part of a face of the user is captured as a response to when the evaluation resulting in a conclusion that acceptable conditions for capturing medically analyzable image data are present. The imaging data comprises at least one of an image and a video stream. Data representing the imaging data is transmitted from the user-operated hand-held medical body-imaging device to an external party.

One advantage with the proposed technology is that with access to images captured under suitable ambient conditions, the analysis of the images becomes easier and the estimated responses become more reliable. Other advantages will be appreciated when reading the detailed description.

### BRIEF DESCRIPTION OF THE DRAWINGS

The invention, together with further objects and advantages thereof, may best be understood by making reference to the following description taken together with the accompanying drawings, in which:

FIG. 1 is a schematic illustration of an embodiment of a user-operated hand-held medical body-imaging device;

FIG. 2 is an illustration of illumination directions;

FIG. 3 is a schematic illustration of a backside of the embodiment of a user-operated hand-held medical body-imaging device of FIG. 1;

FIG. 4 is a flow diagram of steps of an embodiment of a method for analyzing a face of a user;

FIG. 5 is a part flow diagram of an embodiment of step S30 of FIG. 4;

FIG. 6 is a schematic illustration of side illumination of a user face;

FIG. 7 is a schematic illustration of different camera coverages of a user face;

FIG. 8 is a schematic illustration of a partial side image of a user face;

FIG. 9 is a part flow diagram of an embodiment of a step of capturing imaging data; and

FIG. 10 is a part flow diagram of an embodiment of a step of post-capturing image quality check.

### DETAILED DESCRIPTION

Throughout the drawings, the same reference numbers are used for similar or corresponding elements.

For the purpose of this application and for clarity, the following definitions are made:

The term “central server” refers to a computer which is available through one or more communication protocols such as the internet.

The term “imaging data” comprises an image or a video stream or both. The term “video stream” is defined as a plurality of images taken by the same camera with high frequency. A video stream can also be denoted a stream of images, and a sequence of images. A video stream for the purpose of pupillometry usually comprise 10-100, or up to 1000 or even more images captured during 0.5-20 seconds. A video stream is normally captured at between 2 frames per second (fps) to 100 fps.

The term “medical body-imaging device” refers to a device for capturing an image or a video stream of a part of a body, to be used for medical purposes. The body is typically the body of the operator operating the medical body-imaging device. The purpose for this imaging is to determine a condition of the user. The purpose for this imaging is typically of medical character in the sense that some kind of medical condition is evaluated. It could e.g. be used to indicate if the user has been taking drugs recently. It could alternatively determine if the user has had a concussion. Another example is that the imaging is used for diagnosing neurological disease. Yet another example is imaging for registration of body characteristics to be sent to a distant physician. The consequences of the estimated medical condition can be of very different nature, ranging from imprisonment, for the user taking drugs, sick leave, for the user with confirmed concussion, to care efforts, for the user with a neurological disease, to treatment recommendations, for the registration of body characteristics, as non-limiting examples.

One of the medical body-imaging processes that may be used in the above-mentioned medical body-imaging device is pupillometry. The term “pupillometry” is defined as the study of pupils with or without stimuli. Pupillometry includes, but are not limited to, the measurement, study and interpretation of the pupillary size, the pupillary light reflex, the ability of an individual to cross eyes, i.e. non-convergence, and the involuntary type tremors known as nystagmus, to mention a few non-limiting examples.

For a better understanding of the proposed technology, it may be useful to begin with a brief overview of an example scenario. From an overview level, there is a user who intends, voluntary or compulsory, to conduct pupillometry, for example for determining the pupil response to light. The user who shall conduct pupillometry, for example determining pupil response to light, is provided with a device. Upon starting the device, it is requested that imaging procedures are performed at such illumination conditions that ensures good possibilities for analysis. The device is intended to be used in everyday life, meaning at any time of the day and at any location, irrespective of ambient light conditions at the time of use.

In cases where the user voluntary performs the tests, it might be difficult for an un-experienced user to find adequate illumination conditions. In the case the testing is not voluntary, e.g. when an addict is requested to perform repeated tests e.g. according to a test schedule, in order to prove a non-intoxication state, imaging at un-sufficient illumination conditions might even be a strategy for giving inconclusive results. There is thus a need for a guiding of unexperienced/resistant users to reach good recording conditions before the actual imaging is performed.

According to the present technology, the medical body-imaging device itself is used for providing a guiding of the user to acceptable illumination conditions.

In the above indicated scenario, the medical body-imaging device provides instructions to the user on how to proceed in order to make a high-quality measurement of the pupils. When conditions are such that a high-quality image can be captured, the medical body-imaging device captures an image or a video stream of images, depending on the purpose. Thereafter, the captured image or stream of images is assessed for quality. If the quality is sufficient, the results of the image or the captured stream can be released and used for the intended purpose. The present technology relates to the process of ensuring quality, i.e. a method and/or device for ensuring adequate ambient conditions and the quality control of the captured image or video stream.

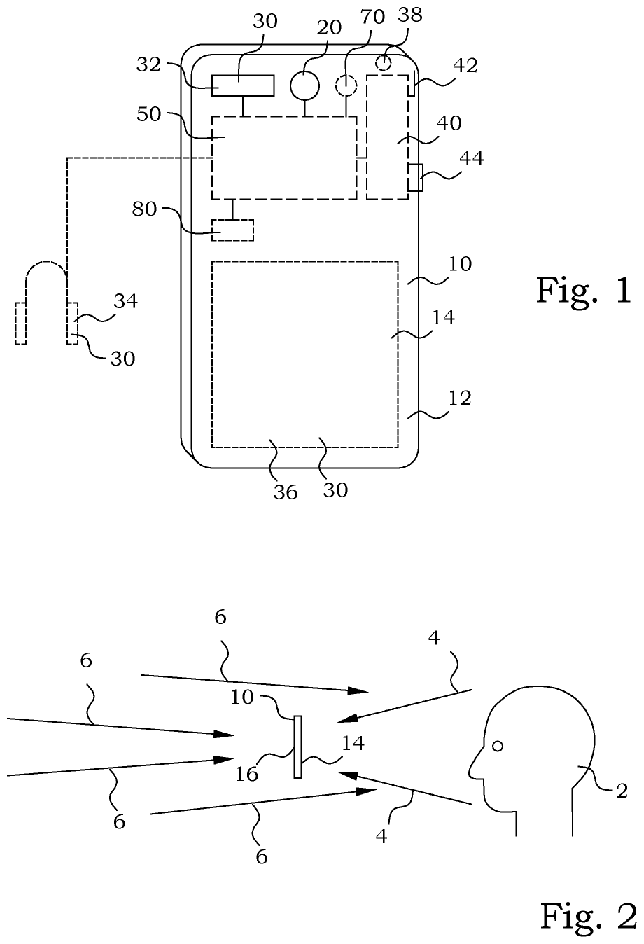

FIG. 1 illustrates schematically an embodiment of a user-operated hand-held medical body-imaging device 10. The user-operated hand-held medical body-imaging device 10 can be a dedicated device for this purpose, or, as in the illustrated embodiment, based on a smart phone 12. The user-operated hand-held medical body-imaging device 10 comprises a primary camera 20. The primary camera 20 is configured for capturing imaging data of at least a part of a user face and for providing pre-capturing image information as experienced in a first direction, typically in a direction from the user towards the primary camera 20. This primary camera 20 is thus typically provided at a front side 14 of the user-operated hand-held medical body-imaging device 10, e.g. the smart phone 12. The imaging data comprises, as mentioned above, at least one of an image and a video stream. Preferably, the primary camera 20 has a resolution of at least 2 Mpixel. In cases a video stream is recorded, the video stream has preferably a duration of a at least 1s. The pre-capturing image information comprises ambient illumination level as experienced in the first direction.

The user-operated hand-held medical body-imaging device 10 further comprises user-interaction means 30. The user-interaction means 30 is configured for providing information to the user. In preferred embodiments, the user-communication means 30 can be one or more of a speaker, a head-phone, a display and a vision indicator. In the present embodiment, the user-communication means 30 is a loudspeaker 32. However, as indicated by dotted lines, the user-communication means 30 may be a pair of headphones 34, or a display 36 or an indicator lamp 38, or a combination of these.

The user-operated hand-held medical body-imaging device 10 further comprises data communication means 40. The data communication means 40 is configured for external data communication. In different embodiments, the data communication means 40 may be one or more of a wireless communication unit 42 and a terminal 44 for electrical and/or optical wire connections. This data communication means 40 may, when the user-operated hand-held medical body-imaging device 10 is based on a smart phone 12, be the ordinary communication means of the smart phone 12.

The user-operated hand-held medical body-imaging device 10 further comprises processing means 50. This processing means 50 may be considered as the central feature in the user-operated hand-held medical body-imaging device 10, coordinating the operations of the other parts. To this end, the processing means 50 is connected to the primary camera 20, the user-communication means 30 and the data communication means 40. In the case of using a smart phone 12, this processing means 50 may comprise parts of the inherent smart phone processors, supported by adequate dedicated software. The processing means 50 is thereby configured for receiving the pre-capturing image information from the primary camera 20. The processing means 50 is furthermore configured for evaluating if acceptable conditions for capturing medically analyzable image data are present. No communication with any external devices is therefore necessary in order to obtain such evaluations, which ensures that appropriate measurements can be performed also at locations where external communication is absent or of low quality. This evaluation is based on the pre-capturing image information. The processing means 50 is furthermore configured for communicating operation instructions to the user in response to the evaluation. The communication of operation instructions is performed using the user-communication means 30. In the present embodiment, using a loudspeaker, pre-recorded voice instructions or synthetically composed voice instructions can be provided as the operation instructions. This interaction with the user herself/himself is important for enabling for instance adjustments in relative positions between the user and the user-operated hand-held medical body-imaging device 10.

This evaluation followed by the operation instructions enables the user-operated hand-held medical body-imaging device 10 to provide the user with adequate instructions how to obtain acceptable conditions for capturing medically analyzable image data. This will be discussed in further detail below.

When appropriate conditions are found, recording may take place. To this end, the processing means 50 is further configured for recording the imaging data from the primary camera 20 and for transmitting data representing the imaging data using the data communication means 40.

FIG. 2 illustrates schematically illumination conditions in connection with a user 2 using a user-operated hand-held medical body-imaging device 10. Light that is impinging on the user-operated hand-held medical body-imaging device 10 from the first direction 4. This light represents the light emitted from the part of the user 2 that is intended to be imaged as well as ambient light from e.g. the background. This light in the first direction 4 reaching the user-operated hand-held medical body-imaging device 10 gives some indication about the general illumination level of the user 2 and the surroundings. This information can, as described above be used for evaluating if adequate illumination conditions prevail.

However, such illumination analysis may be further improved if also the light in an opposite direction 6 is considered. Light in the opposite direction 6 impinges on the backside of the user-operated hand-held medical body-imaging device 10 as well as on the surface of the face of the user 2 that is to be imaged. By comparing the illumination in the two directions, information such as light contrast as well as a better estimation of a general illumination level can be achieved. This is discussed in further detail below.

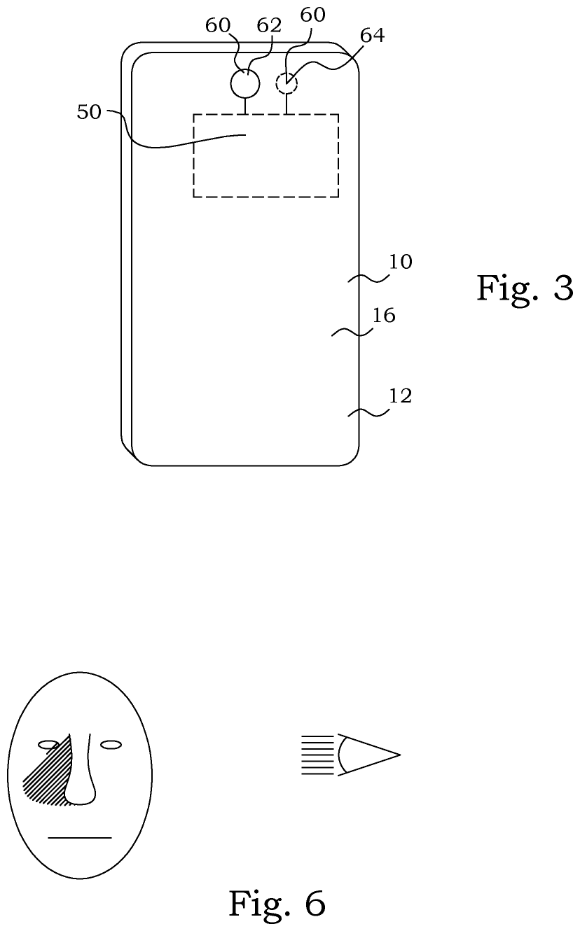

FIG. 3 illustrates schematically the embodiment of a user-operated hand-held medical body-imaging device 10 of FIG. 1 seen from the back-side 16. The user-operated hand-held medical body-imaging device 10 further comprises a light meter 60. The light meter 60 is configured for providing pre-capturing image information comprising ambient illumination level as experienced in a second direction, opposite to the first direction mentioned further above. In the particular embodiment of FIG. 3, the light meter 60 is a secondary camera 62. Alternatively, or as a complement, the light meter 60 can be an ambient illumination level detector 64, as illustrated by the dotted lines. In other words, the light meter 60 is selected as at least one of a secondary camera 62 and an ambient illumination level detector 64.

The light meter 60 is connected to the processing means 50. Thereby, the processing means 50 is further configured for receiving the pre-capturing image information from the light meter 60. The processing means 50 is furthermore configured for performing the evaluation if acceptable conditions for capturing medically analyzable image data are present further based on the pre-capturing image information from the light meter 60.

In a preferred embodiment, the processing means 50 is configured for performing the evaluation if acceptable conditions for capturing medically analyzable image data are present based on a homogeneity measure of ambient light, deduced from the pre-capturing image information from the primary camera as well as the pre-capturing image information from the light meter 60.

The analysis of the light conditions may be further improved by actually utilizing the imaging possibilities of the primary camera. By recording a pre-capturing image, image quality quantities, such as e.g. contrast, average illumination level, shadowing, sharpness etc. can be evaluated and used for providing the user with adequate operational instructions. The pre-capturing image can also be used for evaluating if the intended parts of the user face is imaged, if the imaged area is adequate and if the imaging is made in a suitable angle. If e.g. the user eyes are to be imaged, it can be concluded if the eye are in the image, if both the eyes are imaged simultaneously and if the angle is such that e.g. shadowing by the nose is avoided.

Therefore, in a preferred embodiment, the pre-capturing image information from the primary camera further comprises an image taken opposite to the first direction. The processing means is therefore further configured for performing the evaluation if acceptable conditions for capturing medically analyzable image data are present based on analysis of the image received from the primary camera.

Preferably, the processing means is further configured for deducing image properties from the image received from the primary camera, selected as one or more of an imaged part of user face, a coverage area at user face, an angle between user body and the primary camera, sharpness of the image and light contrast within image. The processing means is further configured for performing the evaluation if acceptable conditions for capturing medically analyzable image data are present based on the deduced image properties.

In a preferred embodiment, the device used for the capture of an image or a video stream of the user shall have at least one primary camera directed in a first direction and at least one light sensing device sensing in a second direction. The first and second directions are essentially opposite directions. The light sensing device may be a secondary camera. For the purpose of capturing images of the majority of the face of the user and in particular the eye-region of the user, the primary camera should preferably have a resolution of at least 2 mega pixels, such as 1920*1080 pixels, so as to allow an image with sufficient resolution of the complete face of the user. The device shall preferably have an embedded computer chip as the processing means to enable internal computations and to enable communication with an external party, e.g. a central server, possibly located in the cloud. The device shall preferably have means for generating audio for communication with the user. The device shall preferably be of a size and weight that allows an average person to carry it using only one hand. This means that the device preferably shall occupy a volume smaller than 0.5 litres, it shall preferably have a weight less than 0.5 kg, and shall preferably have a characteristic length of less than 30 cm, measured in any direction. The device is typically a smartphone.

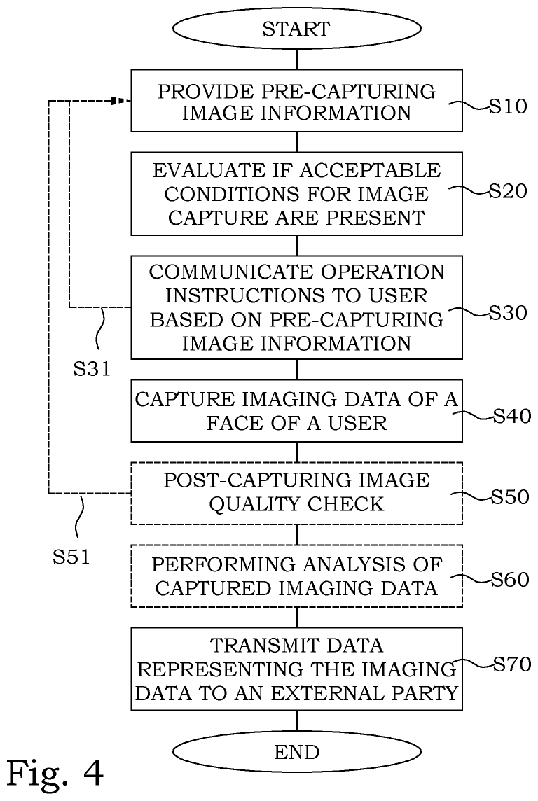

FIG. 4 is a flow diagram of steps of an embodiment of a method for analyzing a face of a user. In step S10, pre-capturing image information is provided. This pre-capturing is performed in a user-operated hand-held medical body-imaging device. The pre-capturing image information comprises ambient illumination level as experienced in a first direction towards the user. In step S20, it is evaluated, in the user-operated hand-held medical body-imaging device, if acceptable conditions for capturing medically analyzable image data are present based on the pre-capturing image information.

In a preferred embodiment, the pre-capturing image information further comprises ambient illumination level as experienced in a second direction, opposite to the first direction. The evaluating step S20 is thereby performed further based on the ambient illumination level as experienced in the second direction.

In a further preferred embodiment, evaluating step S20 is based on a homogeneity measure of ambient light, deduced from the ambient illumination level as experienced in the first direction as well as the ambient illumination level as experienced in the second direction.

In step S30, operation instructions are communicated from the user-operated hand-held medical body-imaging device to the user in response to the evaluation of step S20. Depending on the outcome of the evaluation in the evaluating step S20, the operation instructions may comprise the instruction to obtain better illumination conditions, whereby the process returns to step S10, as indicated by the dotted line S31. Different details of this step will be discussed more below. The step S30 of communicating operation instructions to the user is preferably performed using at least one of audio signals and visible signals.

In step S40 imaging data of a face of said user is captured as a response to when the evaluation in the evaluating step S20 results in a conclusion that acceptable conditions for capturing medically analyzable image data are present. This capturing is performed in the user-operated hand-held medical body-imaging device. The imaging data comprises one or both of an image and a video stream. Preferably, and in particular when the captured imaging data is intended for pupillometry, the step of capturing S40 imaging data comprises capturing of a video stream having a duration of at least 1 s. The video streams may then be long enough to enable analysis of e.g. pupillary light reflex measurements

In step S70, data representing the imaging data is transmitted from the user-operated hand-held medical body-imaging device to an external party. The transmitted data may comprise the originally captured imaging data and/or analysis thereof. The step S70 of communicating data representing the imaging data is preferably performed using at least one of wired communication and wire-less communication. This communication is not very time-critical, which means that e.g. slow communication due to slow wire-less communication coverage anyway may be acceptable.

Preferably, the pre-image capture phase of quality control is intentionally an iterative process. The device will first assess the current conditions to capture images. If conditions are appropriate, this is communicated to the user. If conditions need improvement, this is continuously communicated to the user, while continuously reassessing the current conditions to capture images. When the user finds a location with adequate conditions, this is communicated to the user.

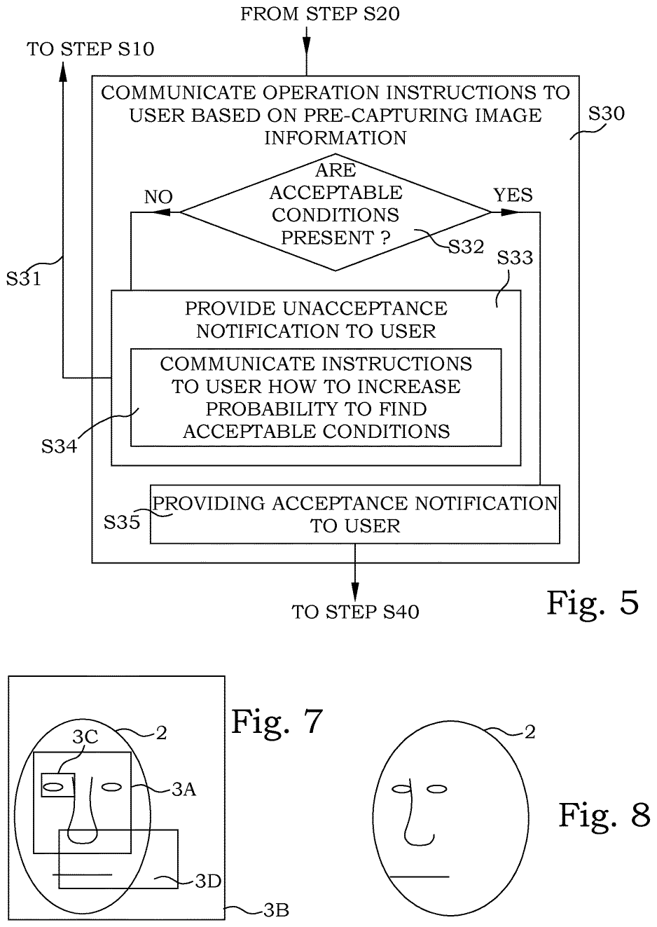

FIG. 5 is a part flow diagram of a preferred embodiment of the step of communicating S30 of FIG. 4. In this preferred embodiment, the step of communicating S30 operation instructions to the user comprises the step S32 in which it is concluded if acceptable conditions for capturing medically analyzable image data are present. If acceptable conditions for capturing medically analyzable image data are not present, i.e. as a response to when the step of evaluating results S20 in a conclusion in step S32 that acceptable conditions for video recording are not present, the process continues to step S33, in which an unacceptance notification is provided to the user. The unacceptance notification informs the user that acceptable conditions for capturing medically analyzable image data are not present.

The meaning of the term “acceptable conditions” depends on the type of measurements performed. As a non-limiting example, for generic pupillometry where eye movements are evaluated, acceptable conditions means that the eyes are located in the image and that there is sufficient light to clearly distinguish the iris from the whites of the eye (sclera). This light level corresponds to about 30 lux with current cameras in mobile phones.

As another non-limiting example, for measurement of the pupillary light reflex, acceptable conditions are stricter. The pupillary light reflex is evaluated by measuring pupil size before and after an incident light pulse. To do this adequately, acceptable conditions comprise the following. Eyes must be located in the image. Illumination must be even across the eyes, which for example could be violated by the nose placing one eye in a shadow. There should be sufficient light to distinguish the pupil from the iris, which corresponds to about 30 lux with current cameras in mobile phones. For pupillary light reflex measurements, there should also be less light than about 1500 lux. At this level, the ambient light has forced the pupil to contract to its smallest size, leaving no room for the pupil to react on an additional light pulse.

Preferably, as indicated by step S34, the unacceptance notification comprises instructions to the user how to change position of the user-operated hand-held medical body-imaging device or ambient light conditions to increase a probability to find acceptable conditions for capturing medically analyzable image data. The process than returns to step S10 as illustrated by the arrow S31.

The step S34 typically comprises two types of instructions. A first type of instruction is related to light quantity and light homogeneity. For such purposes, the image analysis can be applied to instruct the user to change a position of the user together with the user-operated hand-held medical body-imaging device to find more appropriate ambient light conditions. A second type of instruction is related to the object to be imaged. For such purposes, the image analysis can be applied to instruct the user to move the user-operated hand-held medical body-imaging device relative to the body to place the region of interest in the center of the image.

In the preferred embodiment of FIG. 5, the step S30 of communicating operation instructions to the user also comprises step S35, in which an acceptance notification is provided to the user. This step is performed if it in step S32 is found that the step of evaluating S20 resulted in a conclusion that acceptable conditions for capturing medically analyzable image data are present. The acceptance notification confirms that acceptable conditions for capturing medically analyzable image data are present.

Preferably, the step of capturing imaging data S40 is initiated as a response when the step of evaluating S20 results in a conclusion that acceptable conditions for capturing medically analyzable image data are present.

In the above-described pre-image capture phase of quality control, the absolute ambient light condition is highly relevant for capturing high-quality images, e.g. for capturing high-quality images of the eyes of the user. The absolute ambient light condition can be estimated by taking an image with one of the available cameras. Absolute ambient light conditions include, but are not limited to, average light quantity, for example too strong light, or too low light. Unfavourable absolute light conditions can be communicated to the user together with an instruction on how to find a location with better absolute ambient light conditions, as indicated above.

In the pre-image capture phase of quality control, the homogeneity of the ambient light conditions are also useful for capturing high-quality images, e.g. for capturing high-quality images of the eyes of the user. The homogeneity of the ambient light conditions can be estimated by comparing images taken with both a primary camera in the first direction and a secondary camera or other light detector means in the second direction. If the homogeneity of ambient light conditions is unfavourable, for example too strong light on one side and too low light on the opposite side, it can be communicated to the user together with an instruction on how to find a location with better homogeneity of ambient light condition, as indicated above.

In an apparatus view, e.g. with reference to FIG. 1, the processing means 50 is further configured for providing an unacceptance notification to the user informing the user that acceptable conditions for capturing medically analyzable image data are not present, as a response to when the evaluation results in a conclusion that acceptable conditions for capturing medically analyzable image data are not present. Preferably, the unacceptance notification to the user comprises instructions to the user how to proceed to increase a probability to find acceptable conditions for capturing medically analyzable image data.

Preferably, the processing means 50 is configured for providing an acceptance notification to the user confirming that acceptable conditions for capturing medically analyzable image data are present, as a response to when the evaluation results in a conclusion that acceptable conditions for capturing medically analyzable image data are present.

Preferably, the processing means 50 is further configured to control the primary camera 20 to initiate the capturing of the imaging data as a response to when the evaluation results in a conclusion that acceptable conditions for capturing medically analyzable image data are present.

If an additional light source, physically connected to an imaging equipment, is dedicated for providing adequate light conditions, the relation between the imaging equipment and the light source is known and adjustments can easily be performed automatically and even remotely. However, using ambient light for body imaging demands further considerations to be made.

A particularly important aspect of ambient light homogeneity is detrimental effects originating from light source positioning. The relative position between the light source(s), the user and the imaging equipment is of interest. Adjustments of such relative positions have to be performed by the user himself/herself and a user-interaction means is therefore necessary to be present. An external and distant light source which is inadequately placed may introduce shadows in the face of the user during image capture. One such example, as illustrated in FIG. 6, is with a light that comes essentially from the side. This may e.g. be the case during sunrise, when the light from the sun may illuminate the face of the user from the side, illuminating one eye and leaving the other eye in the shadow behind the nose. In this case, the eyes of the user are exposed to a heavily imbalanced lighting condition, which in turn makes image analysis complicated. For the purpose of pupillometry, the homogeneity of the ambient light conditions must be determined in a manner that includes the confirmation that detrimental effects from light source positioning are sufficiently small.

The homogeneity of the ambient light conditions is particularly relevant when evaluating the pupillary light reflex. A practical effect of inadequate light source positioning may be that in a situation where the average light conditions are acceptable. The user may under such circumstances still be instructed to change position so as to reduce the effects of shadows and the similar in the area of medical relevance, which is the eyes in this particular example.

The usefulness of the pre-capture phase is further increased by utilizing not only intensity measures from the primary camera, but also other properties of images taken by the primary camera. In other words, preferably, the pre-capturing image information further comprises an image taken opposite to the first direction, whereby the evaluating step S20 (FIG. 4) is based on an analysis of the image.

In such ways, the analysis of the image may comprise deducing of image properties from the image, selected one or more of an imaged part of the user face, a coverage area at user face, an angle between user body and the user-operated hand-held medical body-imaging device, sharpness of the image and light contrast within image.

In the pre-image capture phase of quality control, the orientation of the device is important for capturing high-quality images of the eyes of the user. This involves locating the device at a suitable distance from the object, i.e. the face of the user, and at an angle which allows the camera to capture the majority of the face. In particular, both eyes of the user must be part of the captured image. The adequacy of the orientation of the device can be determined by taking a test image, using an artificially intelligent face recognition module locate the face and the eyes of the user as captured in the test image, and determine if a sufficient portion of the face and the eyes are visible in the test image. Supporting a user with orientation is particularly important if the camera used on the device is facing in a different direction than the display of the device, i.e. when the display is not visible to the user. If there is insufficient coverage of the face and the eyes in the test image, instructions for how to improve the orientation of the device can be communicated to the user.

As an example, if pupillometry is to be performed, the majority of the face and in particular the eye-region is requested to be covered by the primary camera, however, not by a too large margin. In FIG. 7, a user 2 face is shown. A primary camera covering the area 3A may be appropriate to enable a simultaneous pupillometry of both eyes. A camera coverage area as illustrated by the rectangle 3B may still be useful but gives a lower resolution. A camera coverage area as illustrated by the rectangle 3C is insufficient for enabling pupillometry of both eyes simultaneously. A camera coverage area as illustrated by the rectangle 3D is misdirected to a different face part and is also useless for pupillometry. An imaged part of the user face, and a coverage area at user face is obviously both of interest for improving a quality of the images or videos to be taken.

In FIG. 8, a face of a user 2 is illustrated in an angled view. Such an image may also be difficult to use for pupillometry, since the conditions for the two eyes are different. An angle between user body and the user-operated hand-held medical body-imaging device may therefore also be of interest to control to optimize the images or videos to be taken.

The sharpness of the image may be a factor that may influence the quality of the finally captured image or video. Sharpness may be evaluated from a camera pre-capture image. One factor that influences the sharpness is vibration. If the user has a steady hand, the sharpness may be sufficient. If the user is trembling, the user may seek for a support for the user-operated hand-held medical body-imaging device in addition to the hand for mitigating a blurred image. In other words, in the pre-image capture phase of quality control, the steadiness of the position in which the device is located will impact image quality. In practice, a user who is holding a smartphone in one hand may not necessarily be steady enough for image capture to be of sufficient quality. The steadiness of the device may be assessed either through image analysis, locating motion blur, or through reading an accelerometer in the device during the pre-image capture phase. Poor steadiness can be communicated to the user together with an instruction on how to make the image capture situation adequate from a steadiness perspective. In one embodiment, the user-operated hand-held medical body-imaging device 10, e.g. according to FIG. 1, therefore comprises an accelerometer 80. The accelerometer 80 is connected to the processing means 50, and wherein the processing means 50 is further configured for providing the information to the user in further response to the readings of the accelerometer 80.

In a method view, the step of providing pre-capturing image information S10 preferably further comprises the step of measuring an acceleration of the user-operated hand-held medical body-imaging device, whereby acceleration measures are to be used in the step of evaluating S20 as a representation of movement blurring.

When adequate light conditions have been confirmed, user-operated hand-held medical body-imaging device captures an image or a sequence of images. The adequate light conditions may have to be confirmed by the user, or the capturing may be started automatically. In the case of pupillometry, the capturing involves the eye-region of the face of the user and may be performed according to any procedure known in the art, e.g. similar to what is described in U.S. Pat. No. 6,116,736. The location, size and shape of the pupil is calculated for each eye and captured image, producing results similar to what has been described in the past (such as in Wallace B. Pickworth, Reginald V. Fant, and Edward B. Bunker 1998; Chapter 4.3 Effects of Abused Drugs on Pupillary Size and the Light Reflex in In Drug Abuse Handbook editor S. B. Karch CRC-Press 1998 and Concussion Mckay, (2020) 5(4), CNC82, https://doi.org/10.2217/cnc-2020-0016]).

In other words, in one embodiment, the step of capturing S40 (FIG. 4) imaging data comprises capturing of imaging data of a face of the user.

In an apparatus view, the part of the user body captured by the primary camera is a face of the user. The imaging data thereby enables pupillometry. Preferably, the imaging data covers both pupils of the user, whereby simultaneous pupillometry of both eyes is enabled.

The analysis may be performed at an external node, in which case the imaging data has to be transmitted to this external node. Alternatively, as illustrated by step S60 in FIG. 4, analysis of captured imaging data may be performed by the user-operated hand-held medical body-imaging device, e.g. by the processing means. In particular, in one embodiment, the method for analyzing a face of a user further comprises the step of performing pupillometry using the imaging data. Preferably, the imaging data of the face of the user covers both pupils of the user. The step of performing pupillometry then preferably comprises performing of simultaneous pupillometry of both eyes.

The response behaviour of a pupil that is exposed for changing light conditions may reveal much information about the user condition. To this end, in one embodiment, e.g. with reference to FIG. 1, the user-operated hand-held medical body-imaging device further comprises illumination means 70 for visible light, aligned with the primary camera and configured to expose the face of the user to visible light pulses. In other words, the user-operated hand-held medical body-imaging device shall preferably be equipped with a light generating item which illuminates in the second direction, such as a LED emitting visible light. The processing means is consequently preferably further configured for controlling the light pulses during a duration of the capturing of the imaging data. This thereby enabling pupillary light reflex measurements. For pupillary light reflex measurements to produce medically relevant results, the eyes have to be illuminated in a homogenous manner.

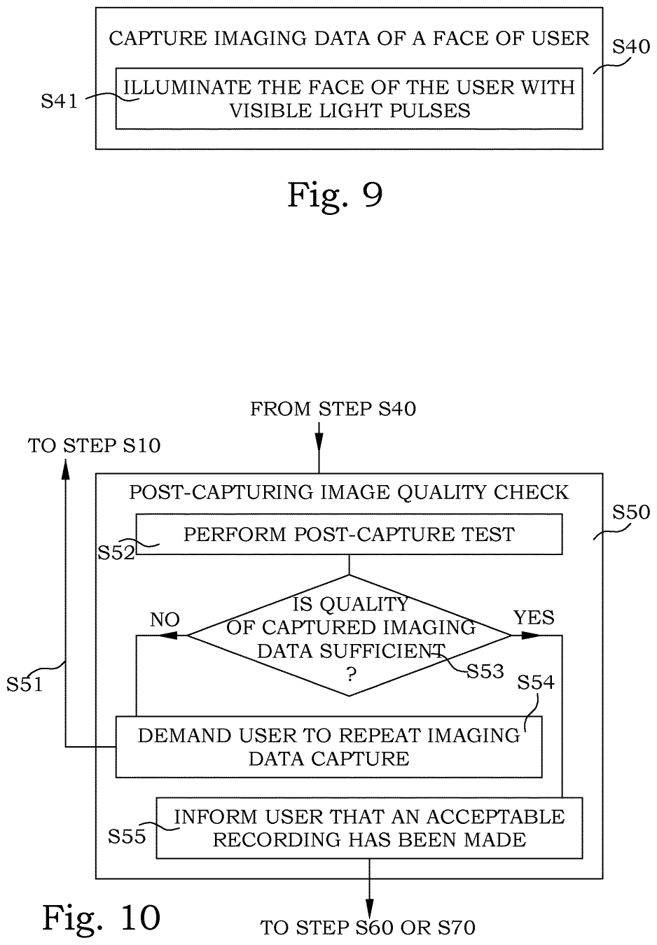

In FIG. 9, an embodiment of step S40 is illustrated, in which the face of the user is illuminated with visible light pulses concurrently to or as a part of the step of capturing imaging data. The step of performing pupillometry then preferably comprises performing pupillary light reflex measurements.

In one embodiment, when the image capture and data extraction is completed, a post-image capture phase of quality control may commence. With reference to FIG. 4, in step S50, a post-capturing image quality check is performed. Here, the captured sequence of images is subjected to analysis to determine if image quality and the quality of the associated extracted features, such as pupil diameter, pupil area, similarity of the pupil to an ellipse, pupil to iris ratio, to mention a few non-limiting examples, are sufficient.

In FIG. 10, an embodiment of the step S50 is illustrated. In step S52 a post-capture test is performed. The post-capture test may in a preferred embodiment be conducted in two stages, and comprises a number of computer-implemented tests of image quality. It operates typically without input from the user.

The first stage of the post-capture test investigates the captured images one at a time. The second stage of the post-capture test investigates the collected time-series of images seen as one unit. The second stage can only be conducted if a video stream has been captured.

In the first stage of the post-capture test, each image is subjected to basic quality evaluation, including for example focus. If an image does not have acceptable basic quality, it is discarded.

In the first stage of the post-capture test, extracted features from each image is subjected to basic quality evaluation related to for example pupil shape. Such quality rules could include comparing extracted feature values to a predetermined range of accepted values. If extracted features from an image does not have acceptable basic quality, the image is discarded.

The discarding of the images are typically governed by a predetermined quality level, with which the evaluations are compared.

When the first stage of the post-capture test is completed, the second stage of the post-capture test is initiated. The second stage can only be conducted if a video stream has been captured.

In the second stage of the post-capture test, the time-series of each extracted features is subjected to basic quality evaluation related to number of data points per unit of time, consecutive missing data leading to large gaps in the time-series. If the collected time-series do not have acceptable basic quality, the data is discarded in its entirety.

In the second stage of the post-capture test, the time-series of each extracted features is subjected to quality evaluation related to continuity. It is known that the collected data should produce a continuous curve without discrete jumps. If the collected time-series do not have acceptable quality in terms of continuity, the data is discarded in its entirety.

Also the discarding of the entire data set is typically governed by a predetermined quality level, with which the evaluations are compared.

It is preferred if the post-capture test is performed within a limited amount of time, e.g. within 30 seconds or more preferably less than 10 seconds. If the time for the post-capture test becomes too long, the user may experience the measurements as slow and select other approaches for obtaining body images. The processing is therefore preferably adapted to the computational power of the processing means in the user-operated hand-held medical body-imaging device to give a reasonable processing time.

A data set which has passed post-image capture phase of quality control is considered trustworthy and can be used for the intended purpose.

In the embodiment of FIG. 10, in a step S52, a quality of the captured imaging data is analyzed in the user-operated hand-held medical body-imaging device by performing a post-capture test. In step S53, it is concluded whether the quality of the captured imaging data is sufficient or not. The user is informed about an outcome of the analysis. If the quality of the captured imaging data was insufficient, i.e as a response to when the analysis found that the quality of the captured imaging data was insufficient, the user is messaged that the captured imaging data had a low quality and as illustrated by step S54, a repetition of the capturing procedures is demanded.

Analogously, in step S55, if the quality of the captured imaging data was sufficient, i.e as a response to when the analysis found that the quality of the captured imaging data was sufficient, the user is messaged that the captured imaging data had an accepted quality.

In an apparatus view, in one embodiment, the processor means is further configured for performing a post-capture test comprising analyzing of a quality of the captured imaging data, and for informing the user about a result of the post-capture test.

In a further embodiment, the processor means is further configured for demanding the user to repeat the imaging data capture procedure if the quality of the captured imaging data is below a predetermined level.

In one embodiment, the processor means is further configured for informing the user that an acceptable recording is made if the quality of the captured imaging data is at least equal to a predetermined level and for transmitting data representing the acceptable imaging data using the data communication means.

As mentioned above, the data transmitted to an external party, i.e. the data representing the imaging data may comprise the imaging data itself.

Alternatively, the data representing the imaging data may comprise evaluated data based on the imaging data itself. In case of implementation to pupillometry, the processing means is preferably further configured for evaluating pupil characteristics from the imaging data, whereby the data representing the imaging data comprises evaluated pupil characteristics data.

Even though this invention is described in the context of measuring the pupillometry as examples, the method to quality-assure an imaging situation is useful for other purposes. One such example could be the use of a smartphone to make an image of a skin deficiency, e.g. due to psoriasis, eczema, heavy sunburns or surgical scars, for the purpose of remote medical evaluation. Such out-patient tools where the individual can use consumer grade devices, such as smartphones, and interact with health care providers are becoming increasingly common, both because of the convenience for the patient and for reducing cost for health care. However, there is a risk of using consumer grade devices, where images may not be adequate due to improper ambient conditions. That risk can be mitigated through implementing a pre-image and post-image quality control process in accordance with the present invention. However, probably the perhaps most interesting area of application is still pupillometry.

In one embodiment, the method is applied for simultaneous image capture of both eyes. Capturing both eyes at the same time allows comparisons of the extracted features of the two different eyes, which is a quality improving aspect.

The method is intended for self-use, i.e. supporting a user to conduct pupillometry or other body image analyses without assistance. It is currently common that pupillometry is conducted with the assistance of a health care practitioner, such as a nurse, so that cameras and other devices are managed by the supporting individual during the time when the user is undergoing pupillometry.

Problematic effects of uneven illumination could in some cases be solved using high dynamic range (HDR) imaging. HDR imaging is well known in the art and relies on taking multiple images with different exposure settings within a short time period, and then algorithmically combine the collected images to one balanced, in terms of light, image. As an example, when imaging a face illuminated from one side, the illuminated eye would be represented by a segment in the image with high-light exposure settings, and the eye located in the shadow of the eye would be represented by a segment in the image with low-light exposure settings. HDR technology may be useful in certain applications of pupillometry, but is not useful at all possible circumstances.

### Example 1

This example illustrates the method in the context of a pupil light reflex measurement.

The results used in this example was obtained a smartphone Samsung S21. Similar results have been obtained in other smartphones, for example iPhone 12, iPhone 13 Mini, and Samsung S22. All tested smartphones had cameras with at least 1920*1080 pixel resolution, all had a front and a back camera, and all had a display on the same side as the front camera and an illumination module in the shape of a light emitting diode near the back camera.

The pupil light reflex measurement was implemented as an App in the device. Upon starting the App, it commences a pre-image quality assessment in accordance with the following:

The light level was measured in the front and back directions by taking pictures with front and back cameras. Light quantity was measured using an indirect method which utilized the built-in auto-exposure functionality present in virtually all smartphone cameras, but in particular in all tested smartphone cameras. The exposure parameters (shutter speed, aperture, ISO) that the camera chose for taking the picture were retrieved and entered into a known formula for reflected light. The relationship between the camera settings (shutter speed, aperture, ISO) are related to luminance in the following manner:

$${N}^{2} / t = L * ( K 1 * log ( S ) + S ) / K 2$$

Where, N is the relative aperture, t is the shutter speed, L is the average luminance of the scene, S is the ISO and K1 and K2 are calibration constants. Light levels front/back were checked against predetermined threshold values for too bright/too dark. Light levels front/back were also compared to each other even if both were within absolute thresholds. If the difference was greater than a predetermined value, the light was considered too uneven (too inhomogeneous) in the front-back plane.

Light levels in the right/left plane were assessed by comparing the average luminosity of the eye regions, extracted using artificially intelligent face detection, in the front camera picture. If light was too unidirectional in the right/left plane, one eye region will be bright and the other, in shadow of the nose, will be dark. If the difference is greater than a predetermined value, the light was considered too uneven (too inhomogeneous) in the right/left plane.

While continuously evaluating (1) absolute light level, (2) light homogeneity in both front/back direction and left/right direction, the user received instructions for how to achieve better imaging conditions. The audio messages to the user comprised “Move to a darker position”, “Move away from light sources”, and the similar.

When an appropriate location had been identified, the user was instructed to turn the smartphone. This is necessary for smartphones because the LED lamp is located on the back side of the smartphone. Immediately thereafter, the App tried to locate the face of the user in the back camera while instructing using audio messages comprising “Move phone away”, “move phone closer”, “tilt head backwards”, “turn head left” and the similar. When the app had determined that the majority of the face including the eye region was visible in the view of the back camera, the procedure of conducting a pupillary light reflex measurement was initiated.

### Example 2

This example illustrates a post imaging quality assessment in the context of a pupil light reflex measurement. This example is based on the access of a series of images captured under confirmed adequate light conditions for the purpose of a pupillary light reflex measurement. In practice, it is a continuation of example 1.

The goal of the post-image quality assessment is to achieve the optimum trade-off between quality of measurement and usability. If quality limits are set too strict, the user will not succeed in making measurements, and if quality limits are set too loose, poor data will be used to make critical decisions.

In the provided time-series of images, the feature “pupil” was extracted for each of the two eyes. This was done using an image-analysis machine-learning model but could, for the purpose of the present invention, rely on any known method for extracting the position and shape of the pupil.

Given the feature pupil for each image, the post-image quality control could commence.

In the post-image quality control assessment, an ellipse was first fitted to the feature pupil. Next, the average probability for all pixels located inside the ellipse was calculated. If the average probability of a pixel being part of the pupil was greater than a predetermined value, the determination of the pupil size was deemed acceptable. For the Samsung S21 device, the predetermined value was average (p)>0.8.

When all images had been assessed in terms of acceptable feature extraction (i.e. pupil size in this example), the time series was assessed for quality, one eye at the time. For a time series to be of acceptable quality, it was required that (1) the overall percentage of acceptable images exceeded a predetermined value, (2) the number of consecutive rejected images were less than a predetermined value, and (3) that each phase of the characteristic shape of a pupil reflex measurement were represented with a sufficient number of images.

In one embodiment, a method for improving quality in image capture for the purpose of pupillometry is presented. This method comprises the following steps. A user who intends to conduct pupillometry, for example determining pupil response to light, is provided with a device. The device is programmed to repeatedly do the following:
determine if the surrounding/ambient light conditions relevant to image capture are adequate for capturing high-quality images;
if it is determined that the surrounding/ambient light conditions relevant to image capture are inadequate, instructions on how to improve conditions are provided to the user;
if it is determined that the surrounding/ambient light conditions relevant to image capture are adequate, proceed to image capture.

The device captures an image or a sequence of images suitable for the type of pupillometry the user is conducting. This step may include exposing the user to a stimulus, such as illuminating the eyes of the user. Upon having completed capturing an image or a sequence of images, the quality of the captured image(s) is assessed using an image quality assessment tool. If the image quality is inadequate, the user is provided with instructions to restart the measurement. If the image quality is adequate, images are provided to an algorithm suitable for conduct pupillometry.

The quality control of images comprises two phases. First, there is a pre-image capture phase of ambient light conditions and the like. Second, there is a post-image capture quality procedure to assess the usefulness of the captured images.

Any camera-based system will produce images that in part reflect the ambient light conditions. The present invention relates to a method for obtaining suitable ambient conditions for producing images of the eyes of an individual.

With access to images captured under suitable ambient conditions, the analysis of the images becomes easier and the estimated responses become more reliable. This is particularly important in cases where the image analysis of the eye is conducted for a medical reason, such as diagnostic or prognostic or continuous monitoring reason.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1. A user-operated hand-held medical body-imaging device, comprising:
a primary camera, configured for capturing imaging data of at least a part of a face of a user and for providing pre-capturing image information as experienced in a first direction;
said imaging data comprising at least one of an image and a video stream;
said pre-capturing image information comprising ambient illumination level as experienced in said first direction;
user-interaction means, configured for providing information to said user;
data communication means, configured for external data communication;
processing means, connected to said primary camera, said user-communication means and said data communication means;
said processing means being configured for receiving said pre-capturing image information from said primary camera, for evaluating if acceptable conditions for capturing medically analyzable image data are present based on said pre-capturing image information, and for communicating operation instructions to said user in response to said evaluation, using said user-communication means; and
said processing means being further configured for recording said imaging data from said primary camera and for transmitting data representing said imaging data using said data communication means.

2. The user-operated hand-held medical body-imaging device according to claim 1, further comprising
a light meter configured for providing pre-capturing image information comprising ambient illumination level as experienced in a second direction, opposite to said first direction;
said light meter being selected as at least one of a secondary camera and an ambient illumination level detector;
said light meter being connected to said processing means, whereby said processing means being further configured for receiving said pre-capturing image information from said light meter, and whereby said processing means is configured for performing said evaluation if acceptable conditions for capturing medically analyzable image data are present further based on said pre-capturing image information from said light meter.

3. The user-operated hand-held medical body-imaging device according to claim 1, wherein said imaging data enables pupillometry;
wherein said user-operated medical body-imaging device further comprises
illumination means for visible light, aligned with said primary camera and configured to expose said face of said user to visible light pulses, whereby said processing means is further configured for controlling said light pulses during a duration of said capturing of said imaging data, thereby enabling pupillary light reflex measurements.

4. A method for analyzing a face of a user, comprising the steps of:
providing, in a user-operated hand-held medical body-imaging device, pre-capturing image information;
said pre-capturing image information comprising ambient illumination level as experienced in said first direction towards said user;
evaluating, in said user-operated hand-held medical body-imaging device, if acceptable conditions for capturing medically analyzable image data are present based on said pre-capturing image information;
communicating operation instructions from said user-operated hand-held medical body-imaging device to said user in response to said evaluation;
capturing, in said user-operated hand-held medical body-imaging device, imaging data of at least a part of a face of said user as a response to when said evaluation resulting in a conclusion that acceptable conditions for capturing medically analyzable image data are present;
said imaging data comprising at least one of an image and a video stream; and
transmitting data representing said imaging data from said user-operated hand-held medical body-imaging device to an external party.

5. The method according to claim 4, wherein said pre-capturing image information further comprises ambient illumination level as experienced in a second direction, opposite to said first direction, whereby said evaluating step is performed further based on said ambient illumination level as experienced in said second direction.

6. The method according to claim 5, wherein said evaluating step is based on a homogeneity measure of ambient light, deduced from said ambient illumination level as experienced in said first direction as well as said ambient illumination level as experienced in said second direction.

7. The method according to any of the claim 4, wherein said pre-capturing image information further comprises an image taken opposite to said first direction, whereby said evaluating step is based on an analysis of said image, and wherein said analysis of said image comprises deducing of image properties from said image, selected as at least one of:
imaged part of user face
coverage area at user face;
angle between user body and said user-operated hand-held medical body-imaging device;
sharpness of image; and
light contrast within image.

8. The method according to claim 4, wherein said step of communicating operation instructions to said user comprises providing of an unacceptance notification to said user informing said user that acceptable conditions for capturing medically analyzable image data are not present, as a response to when said step of evaluating results in a conclusion that acceptable conditions for video recording are not present, and said step of providing unacceptance notification comprises communication of instructions to said user how to change position of said user-operated hand-held medical body-imaging device or ambient light conditions to increase a probability to find acceptable conditions for capturing medically analyzable image data.

9. The method according to claim 4, wherein said step of capturing imaging data comprises capturing of imaging data of a face of said user, wherein said method comprises the further the step of:
performing analysis of captured imaging data, and in particular pupillometry using said imaging data.

10. The method according to claim 4, comprising the further steps of:
performing post-capturing test analyzing, in said user-operated hand-held medical body-imaging device, a quality of said captured imaging data; and
informing said user about an outcome of said step of performing the post-capturing test,
wherein said step of informing said user comprises messaging said user that said captured imaging data had a low quality and demanding a repetition of the capturing procedures, as a response to when said step of analyzing found that said quality of said captured imaging data was insufficient.

11. The user-operated hand-held medical body-imaging device according to claim 2, wherein said imaging data enables pupillometry;
wherein said user-operated medical body-imaging device further comprises
illumination means for visible light, aligned with said primary camera and configured to expose said face of said user to visible light pulses, whereby said processing means is further configured for controlling said light pulses during a duration of said capturing of said imaging data, thereby enabling pupillary light reflex measurements.

12. The method according to claim 5, wherein said pre-capturing image information further comprises an image taken opposite to said first direction, whereby said evaluating step is based on an analysis of said image, and wherein said analysis of said image comprises deducing of image properties from said image, selected as at least one of:
imaged part of user face
coverage area at user face;
angle between user body and said user-operated hand-held medical body-imaging device;
sharpness of image; and
light contrast within image.

13. The method according to claim 6, wherein said pre-capturing image information further comprises an image taken opposite to said first direction, whereby said evaluating step is based on an analysis of said image, and wherein said analysis of said image comprises deducing of image properties from said image, selected as at least one of:
imaged part of user face
coverage area at user face;
angle between user body and said user-operated hand-held medical body-imaging device;
sharpness of image; and
light contrast within image.

14. The method according to claim 5, wherein said step of communicating operation instructions to said user comprises providing of an unacceptance notification to said user informing said user that acceptable conditions for capturing medically analyzable image data are not present, as a response to when said step of evaluating results in a conclusion that acceptable conditions for video recording are not present, and said step of providing unacceptance notification comprises communication of instructions to said user how to change position of said user-operated hand-held medical body-imaging device or ambient light conditions to increase a probability to find acceptable conditions for capturing medically analyzable image data.

15. The method according to claim 6, wherein said step of communicating operation instructions to said user comprises providing of an unacceptance notification to said user informing said user that acceptable conditions for capturing medically analyzable image data are not present, as a response to when said step of evaluating results in a conclusion that acceptable conditions for video recording are not present, and said step of providing unacceptance notification comprises communication of instructions to said user how to change position of said user-operated hand-held medical body-imaging device or ambient light conditions to increase a probability to find acceptable conditions for capturing medically analyzable image data.

16. The method according to claim 7, wherein said step of communicating operation instructions to said user comprises providing of an unacceptance notification to said user informing said user that acceptable conditions for capturing medically analyzable image data are not present, as a response to when said step of evaluating results in a conclusion that acceptable conditions for video recording are not present, and said step of providing unacceptance notification comprises communication of instructions to said user how to change position of said user-operated hand-held medical body-imaging device or ambient light conditions to increase a probability to find acceptable conditions for capturing medically analyzable image data.

17. The method according to claim 12, wherein said step of communicating operation instructions to said user comprises providing of an unacceptance notification to said user informing said user that acceptable conditions for capturing medically analyzable image data are not present, as a response to when said step of evaluating results in a conclusion that acceptable conditions for video recording are not present, and said step of providing unacceptance notification comprises communication of instructions to said user how to change position of said user-operated hand-held medical body-imaging device or ambient light conditions to increase a probability to find acceptable conditions for capturing medically analyzable image data.

18. The method according to claim 13, wherein said step of communicating operation instructions to said user comprises providing of an unacceptance notification to said user informing said user that acceptable conditions for capturing medically analyzable image data are not present, as a response to when said step of evaluating results in a conclusion that acceptable conditions for video recording are not present, and said step of providing unacceptance notification comprises communication of instructions to said user how to change position of said user-operated hand-held medical body-imaging device or ambient light conditions to increase a probability to find acceptable conditions for capturing medically analyzable image data.

19. The method according to claim 5, wherein said step of capturing imaging data comprises capturing of imaging data of a face of said user, wherein said method comprises the further the step of:
performing analysis of captured imaging data, and in particular pupillometry using said imaging data.

20. The method according to claim 6, wherein said step of capturing imaging data comprises capturing of imaging data of a face of said user, wherein said method comprises the further the step of:
performing analysis of captured imaging data, and in particular pupillometry using said imaging data.
